(19) United States
(12) Patent Application Publication
(10) Pub. No.: US 2013/0156865 A1
(43) Pub. Date: Jun. 20, 2013

(54) POTENTIATION INDUCED BY PDE4 INHIBITORS IN THE TREATMENT OF LEUKEMIA

(71) Applicant: Chiesi Farmaceutici S.p.A., Parma (IT)

(72) Inventors: Antonio Bonati, Parma (IT); Paolo Lunghi, Parma (IT)

(73) Assignee: Chiesi Farmaceutici S.p.A., Parma (IT)

(21) Appl. No.: 13/714,938

(22) Filed: Dec. 14, 2012

(30) Foreign Application Priority Data
Dec. 16, 2011 (EP) 11194076.3

(51) Int. Cl. A61K 31/4425; A61K 31/203; A61K 31/44; A61K 33/36

(52) U.S. Cl. CPC A61K 31/4425; A61K 33/36; A61K 31/203; A61K 31/44
USPC 424/623; 514/357

(57) ABSTRACT

Certain PDE-4 inhibitors are useful for the treatment of myeloid and linphoyd malignancies.

### CROSS REFERENCES TO RELATED APPLICATIONS

This application claims priority to European Patent Application No. 11194076.3, filed on Dec. 16, 2011, which is incorporated herein by reference in its entirety.

### BACKGROUND OF THE INVENTION

1. Field of the Invention

The present invention relates to the use of phosphodiesterase-4 (PDE4) inhibitors for the treatment of hematological malignancies. In particular the present invention relates to the use of PDE-4 inhibitors for the treatment of myeloid and linphoyd malignancies and to their combination with antitumoral agents.

2. Discussion of the Background

Hematological malignancies are the types of cancer that could affect blood (leukemia), bone marrow, and lymph nodes. Leukemias are classified as either lymphocytic or myeloid, depending on the type of leukocyte affected. In addition, leukemias are classified as either acute, referring to a rapidly progressing disease that involves immature leukocytes (white blood cells), or chronic, referring to a slower proliferation involving the mature ones.

The myeloid leukemias affect white blood cells (myelocytes) that give rise to granulocytes and include chronic myeloid leukemia (CML) acute myeloid leukemia (AML), also called, acute non-lymphocytic leukemia (ANLL) and a sub-type form known as acute promyelocytic leukemia (APL). Instead, lymphocytic leukemias affect the white blood cells that give rise to various types of lymphocytes and include acute lymphocytic leukemia (ALL), chronic lymphocytic leukemia (CLL), also called chronic granulocytic leukemia; and hairy cell leukemia (HCL). The lymphocytic leukemias are sometimes referred to as B-cell leukemia or T-cell leukemia depending upon whether they arise in antibody-producing B cells (HCL, CLL, and some cases of ALL) or in the T cell lymphocytes involved in cell-mediated immunity (some cases of ALL).

Treatment may include radiation therapy, blood and plasma transfusions, bone marrow transplantation and chemotherapy with anticancer drugs such as differentiation inducing agents, e.g. all trans-retinoic acid (ATRA), and arsenic trioxide (ATO). However, a more general use of ATRA and ATO at the clinical level particular in APL, is limited by toxicity and natural or induced resistance.

Some literature also suggests that agents capable of modulating 3′5′-cyclic adenosine monophosphate (cAMP) levels, for instance phosphodiesterase (PDE) inhibitors, might be useful for the treatment of hematological malignancies alone and/or in combination with anticancer drugs. For example, theophylline, a nonspecific methylxanthine PDE inhibitor, has been shown to induce apoptosis in chronic lymphocytic leukemia (CLL) B-lymphocytes in vitro (see Mentz F et al., Br. J. Hematol. 1995, 90-957-959, which is incorporated herein by reference in its entirety).

WO 2004/062671, which is incorporated herein by reference in its entirety, discloses that certain more selective PDE-4 inhibitors such as piclaimilast and roflumilast alone or in combination with differentiation inducing agents could be used for the treatment of neoplasms of lymphoid cells.

WO 2009/147169, which is incorporated herein by reference in its entirety, discloses that the aforementioned PDE-4 inhibitors in combination with retinoic acid and/or an arsenic derivative could be particularly useful for the treatment of acute myeloid leukemia and acute promyelocytic leukemia.

On the other hand, it is known that PDE-4 inhibitors could exhibit sied effects including nausea and emesis. Said side effects could worsen the tolerability of the chemotherapy treatment.

Therefore, there is still a need for safer and more effective therapeutic approaches for the treatment of hematological malignancies.

WO 2008/093188 and WO 2010/089107, which are incorporated herein by reference in their entireties, disclose derivatives of 1-phenyl-2-pyridinyl alkyl alcohols acting as PDE-4 inhibitors endowed with a more selective action toward the “low affinity rolipram” binding site (LPDE4) in comparison to the “high affinity rolipram” binding site (HPDE4), in order to attenuate or avoid the side effects associated with its inhibition. Said derivatives could hence turn out to be safer.

### SUMMARY OF THE INVENTION

Accordingly, it is one object of the present invention to provide novel methods for treating hematological malignancies.

It is another object of the present invention to provide novel methods for treating hematological malignancies which exhibit reduced side effects.

It is another object of the present invention to provide novel methods for treating myeloid and linphoyd malignancies.

These and other objects, which will become apparent during the following detailed description, have been achieved by the inventors' discovery that combinations of:

i) a compound of general formula (I):

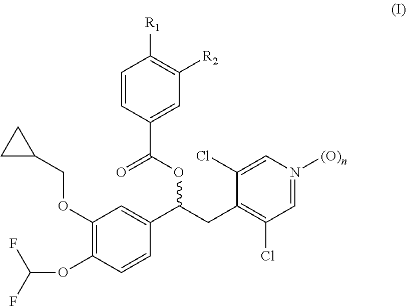

wherein:

n is 0 or 1;

R₁ and R₂ may be the same or different, and are selected from the group consisting of:

linear or branched (C₁-C₆)alkyl, optionally substituted by one or more halogen atoms;

OR₃ wherein R₃ is a linear or branched (C₁-C₆)alkyl optionally substituted with one or more halogen atoms or C₃-C₇ cycloalkyl groups; and

HNSO₂R₄ wherein R₄ is a linear or branched (C₁-C₄)alkyl optionally substituted with one or more halogen atoms,

wherein at least one of R₁ and R₂ is HNSO₂R₄;

ii) a retinoid and/or

iii) an arsenic derivative

are useful for the treatment of hematological malignancies.

Thus, in a first aspect, the present invention provides combinations of:

i) a compound of general formula (I):

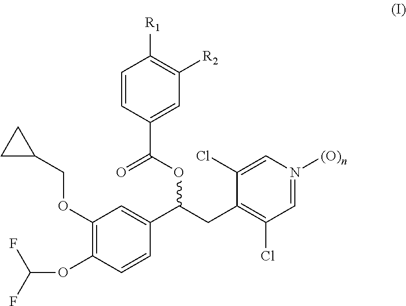

wherein:

n is 0 or 1;

R₁ and R₂ may be the same or different, and are selected from the group consisting of:

linear or branched (C₁-C₆)alkyl, optionally substituted by one or more halogen atoms;

OR₃ wherein R₃ is a linear or branched (C₁-C₆)alkyl optionally substituted with one or more halogen atoms or C₃-C₇ cycloalkyl groups; and

HNSO₂R₄ wherein R₄ is a linear or branched (C₁-C₄)alkyl optionally substituted with one or more halogen atoms,

wherein at least one of R₁ and R₂ is HNSO₂R₄;

ii) a retinoid and/or

iii) an arsenic derivative

for simultaneous, sequential or separate administration.

In a second aspect, the present invention provides a medicament comprising a fixed combination of a compound of general formula (I) with a retinoid and/or an arsenic derivative, and optionally a pharmaceutically acceptable carrier or diluent.

In a third aspect, the present invention provides a kit comprising;

a) a compound of general formula (I), and optionally a pharmaceutically acceptable carrier or diluent in a first unit dosage form;

b) a retinoid and optionally a pharmaceutically acceptable carrier or diluent in a second unit dosage form; and/or

c) an arsenic derivative, and optionally a pharmaceutically acceptable carrier or diluent in a third unit dosage form;

d) container means for containing said first, second and optionally third dosage forms.

In a fourth aspect, the present invention provides a compound of general formula (I) for use for the treatment hematological malignancies.

In a fifth aspect, the present invention provides the use of a compound of general formula (I) for the preparation of a medicament for the treatment of hematological malignancies.

In a sixth aspect, the present invention provides methods for the treatment of hematological malignancies, comprising administering to a patient in need thereof a therapeutically effective amount of a compound of general formula (I).

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the proapoptotic effects of C2 vs Roflumilast (CHF 5152) and Piclamilast (CHF 5889) in combination with Arsenic Trioxide (ATO) on parental (NB4) and arsenic resistant (NB4-AsR) Acute Promyelocytic Leukemia cell lines. Apoptotic cells were detected by flow cytometry using the annexin V-staining method. On the Y-axis the histograms represent the percentage of apoptotic cells measured by Annexin V staining (% ANNEXIN V+) determined for each experimental condition. Vehicle indicates Dimethyl sulfoxide (DMSO); C2, compound C2; 5152, Roflumilast; 5889, Piclamilast; CTR, control; ATO, arsenic trioxide.

FIG. 2 shows the proapoptotic effects of C2 vs roflumilast (CHF 5889) and piclamilast (CHF 5152) in combination with Arsenic Trioxide on different Human Chronic Myelogenous Leukemia cell lines either sensitive or resistant to Imatinib. Apoptotic cells were detected by flow cytometry using the annexin V− or propidium iodide staining methods. On the Y-axis the histograms represent the percentage of apoptotic cells measured by Annexin V staining (% ANNEXIN V+) or sub-G1 population (% sub-G1) determined for each experimental condition. V alues represent the mean of three independent experiments. —SENS indicates sensitive to Imatinib; —RES, resistant to IMATINIB. Vehicle indicates Dimethyl sulfoxide (DMSO); C2, compound C2; 5889, Piclamilast; CTR, control; ATO, arsenic trioxide.

FIG. 3 shows seven Combination Index (IC) plots describing the pharmacologic interactions between C2 and arsenic trioxide (ATO) on seven different human leukemic cell lines. Cell lines were cultured in the presence of escalating doses of C2 (0.5-40 μM), ATO (0.5-5 μM) or combinations of the two agents at a 1:1 ratio (0.5/0.5, 1/1, 1.5/1.5, 2/2 μM) the percentage of apoptotic cells after 72 hours of treatment was evaluated by measurement of sub-G1 DNA content and IC plots were generated using the Calcusyn software: CI less than 1.0 indicates synergism; CI=1.0 indicates additive effect; CI more than 1.0 indicates antagonistic effect. —SENS indicates sensitive to Imatinib; —RES, resistant to Imatinib.

FIG. 4 shows the proapoptotic effects of C2 in combination with Arsenic Trioxide (ATO) in the presence or absence of the specific caspase inhibitors (caspase-8 IN, caspase-9 IN and PANCASPASE IN) on parental (NB4) and arsenic resistant (NB4-AsR) Acute Promyelocytic Leukemia cell lines and Human Chronic Myelogenous Leukemia cell line LAMA-RES resistant to IMATINIB. On the Y-axis the histograms represent the percentage of apoptotic cells measured by Annexin V staining (% ANNEXIN V+). NEG indicates peptide control Cbz-Phe-Ala-fluoromethyl ketone (Z-FA-FMK), CASPASE-8 IN, specific caspase-8 inhibitor Cbz-Ile-Glu(Ome)-Thr-Asp(Ome)-fluoromethyl ketone (Z-IETD-FMK), CASPASE-9 IN, specific caspase-9 inhibitor Cbz-Leu-Glu(Ome)-His-Asp(Ome)-fluoromethyl ketone (Z-LEHD-FMK), PANCASPASE IN, pancaspase inhibitor Cbz-Val-Ala-Asp(Ome)-fluoromethyl ketone (Z-VAD-FMK); CTR, control; C2, compound C2; ATO, arsenic trioxide.

FIG. 5 shows the induction of mitochondrion-mediated apoptosis induced by C2 in combination with Arsenic Trioxide (ATO) on human Chronic Myelogenous Leukemia cell line sensitive (LAMA-SENS) or resistant to Imatinib (LAMA-RES). The histograms represent on the y-axis the percentage of mitochondrion-mediated apoptosis measured by loss of mitochondrial transmembrane potential (LOSS MITOCHONDRIAL) after 48 hours of treatment. CTR indicates control; C2, compound C2; ATO, arsenic trioxide.

FIG. 6 shows the myeloid differentiation induced by C2 vs roflumilast (CHF 5152) and piclamilast (CHF 5889) in combination with all-trans retinoic acid (ATRA) or Arsenic Trioxide (ATO) on parental (NB4) and arsenic resistant (NB4-AsR) Acute Promyelocytic Leukemia cell lines. On the Y-axis the histograms represent the percentage of myeloid differentiation measured either by early expression of CD11b (% CD11b+) or nitroblue tetrazolium reduction test (% NBT+). Vehicle indicates Dimethyl sulfoxide (DMSO); C2, compound C2; 5152, Roflumilast; 5889, Piclamilast; CTR, control; ATRA, all-trans retinoic acid; ATO, arsenic trioxide.

FIG. 7 shows the myeloid differentiation induced by C2 in combination with all-trans retinoic acid (ATRA) on parental (NB4) and arsenic resistant (NB4-AsR) Acute Promyelocytic Leukemia cell lines. On the Y-axis the histograms represent, for each cell line, the percentage of myeloid differentiation measured using a panel of myeloid markers (CD33, CD38, CD11b, CD15, CD14) and nitroblue tetrazolium reduction test (% NBT+). Vehicle indicates Dimethyl sulfoxide (DMSO); C2, compound C2; CTR, control; ATRA, all-trans retinoic acid.

### DETAILED DESCRIPTION OF THE PREFERRED EMBODIMENTS

Unless defined otherwise, all technical and scientific terms used herein have the same meaning as it is commonly understood by the skilled in the art.

The terms “cancer”, “neoplasm,” and “malignancy” are used synonymously.

The terms “LPDE4” and “HPDE4” refer to the two distinct forms in which the enzyme PDE-4 exists. They exhibit different conformations, that were designated as high affinity rolipram binding site or HPDE4, especially present in the central nervous system and in parietal cells, and low affinity rolipram binding site or which is found in the immune and inflammatory cells LPDE4 (see Jacobitz, S et al Mol. Pharmacol, 1996, 50, 891-899, which is incorporated herein by reference in its entirety). While both forms appear to exhibit catalytic activity, they differ with respect to their sensitivity to inhibitors.

The term “halogen atoms” includes fluorine, chlorine, bromine, and iodine.

The expression “linear or branched C₁-C_{X} alkyl” where x is an integer greater than 1, such as C₁-C₆ or C₁-C₄ alkyl, refers to straight or branched chain alkyl groups wherein the number of carbon atoms is in the range 1 to x (e.g. 1 to 6 or 1 to 4). Examples of alkyl groups may thus include methyl, ethyl, n-propyl, isopropyl, t-butyl, pentyl, hexyl, and the like.

Optionally in said groups one or more hydrogen atoms can be replaced by halogen atoms, preferably chlorine or fluorine

The expression “C₃-C₇ cycloalkyl” refers to cyclic non-aromatic hydrocarbon groups containing 3 to 7 ring carbon atoms. Examples of them may thus include cyclopropyl, cyclobutyl, cyclopentyl, cyclohexyl, and cycloheptyl.

The term “retinoids” refers to substances such as (2E,4E,6E,8E)-3,7-dimethyl-9-(2,6,6-trimethylcyclohexen-1-yl)nona-2,4,6,8-tetraenoic acid, commonly known as all-trans retinoic acid or a cis derivative such as 9-cis retinoic acid or 13-cis retinoic acid.

The term “arsenic derivative” includes three inorganic forms: arsenic disulfide, arsenic trisulfide, and arsenic trioxide.

Unless otherwise provided, when referring to chiral compounds, a degree of purity “substantially pure” means at least greater than about 97% chirally pure, preferably greater than 99% and most preferably greater than 99.9%.

“Daily dose” means the overall quantity of active substance administered during the day. Said daily dose may be administered in one or more unit doses.

The term “fixed combination” means a combination wherein the active substances are in a constant quantitative ratio, i.e. that the amount of each active substance does not change.

The term “pharmaceutical acceptable” refers to an excipient or carrier that does not produce an allergic or similar untoward reaction when administered to a patient.

The term “an effective amount of a compound for treating a particular disease” is an amount that is sufficient to ameliorate, or in some manner reduce, the symptoms associated with the disease.

The term “treatment” means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “synergistic” means that the activity of the two compounds is more than would be expected by summing their respective individual activities in a given assay.

Thus, the present invention provides a combination of a compound of general formula (I) acting as inhibitor of the phosphodiesterase-4 (PDE4) enzyme with a retinoid and/or an arsenic derivative.

It has been found that a representative member of the compounds of general formula (I) in combination with arsenic trioxide (ATO) in a human model of acute promyelocytic leukemia resistant to ATO, is significantly more effective than the coresponding combinations with PDE-4 inhibitors such as roflumilast and piclamilast (see Example 1 below).

Also in chronic myeloid leukemia cell lines resistant to Imatinib, said PDE-4 inhibitor turned out to be capable of enhancing the cytotoxic effect of ATO with doses 5-10 times lower than those of piclamilast (see Example 2 below).

Moreover, the same representative compound has also been found capable of enhancing the cytodifferentiating properties of ATO in sensitive acute promyelocytic leukemia cell lines sensitive or resistant to ATO, with doses 5 to 10 times lower than those of piclamilast or roflumilast (see Example 6 below).

The PDE-4 inhibitors of general formula (I) appear to strongly potentiate the cytodifferentiation effects of retinoids, in particular ATRA, in either in ATO-sensitive or ATO-resistant APL cell lines, making possible to reduce the doses of the differentiation inducing agent without affecting the therapeutic effect (see Example 7 below). This would be a great advantage in terms of safety and tolerance from the patients as the conventional dose of ATRA (20 mg day corresponding to about 3.0-3.5 mg/kg day) may be reduced up to half of the conventional dose when combined with a compound of general formula (I).

The compounds of general formula (I) are disclosed in WO 2010/089107, which is incorporated herein by reference in its entirety, and may be prepared according to method therein disclosed.

It will be apparent to those skilled in the art that said compounds at least contain one asymmetric center, and therefore exist as optical stereoisomers. The invention encompasses both the (+)- and the (−)-enantiomers, but the compounds of formula (I) which are (−) enantiomers with configuration (S) at the chiral center are preferred. More preferably, said enantiomer shall be utilized in a substantially pure chiral form.

Preferred groups of compounds of general formula (I) are those wherein:
R₁ is HNSO₂R₄, R₂ is OR₃ and n is 0;
R₁ is HNSO₂R₄, R₂ is OR₃ and n is 1;
R₁ is HNSO₂R₄, wherein R₄ is methyl, R₂ is OR₃, wherein R₃ is cyclopropylmethyl and n is 0;
R₁ is HNSO₂R₄, wherein R₄ is methyl, R₂ is OR₃, wherein R₃ is cyclopropylmethyl and n is 1;
R₁ is linear or branched C₁-C₆ alkyl, R₂ is HNSO₂R₄ and n is 0;
R₁ is methyl, R₂ is HNSO₂R₄, wherein R₄ is methyl and n is 0;
R₁ is linear or branched C₁-C₆ alkyl, R₂ is HNSO₂R₄ and n is 1;
R₁ is methyl, R₂ is HNSO₂R₄, wherein R₄ is methyl and n is 1;
R₂ is linear or branched C₁-C₆ alkyl, R₁ is HNSO₂R₄ and n is 0;
R₂ is methyl, R₁ is HNSO₂R₄, wherein R₄ is methyl and n is 0;
R₂ is linear or branched C₁-C₆ alkyl, R₁ is HNSO₂R₄ and n is 1;
R₂ is methyl, R₁ is HNSO₂R₄, wherein R₄ is methyl and n is 1;
R₁ is OR₃, R₂ is HNSO₂R₄ and n is 0;
R₁ is OR₃, R₂ is HNSO₂R₄ and n is 1;
R₁ is OR₃ wherein R₃ is cyclopropylmethyl, R₂ is HNSO₂R₄ and R₄ is methyl and n is 1;
R₁ is OR₃, R₂ is HNSO₂R₄ and n is 1;
both R₁ and R₂ are HNSO₂R₄ and n is 0;
both R₁ and R₂ are HNSO₂R₄, wherein R₄ is methyl and n is 0;
both R₁ and R₂ are HNSO₂R₄ and n is 1;
both R₁ and R₂ are HNSO₂R₄, wherein R₄ is methyl and n is 1.

According to a even more preferred embodiment of the invention, the compound of general formula (I) is selected from the compounds C1, C2, C3, C4, C5, and C6 shown in the following Table.

TABLE

| Compound | Chemical name |
|---|---|
| C1 | (-)-3-Cyclopropylmethoxy-4-methanesulfonylamino-benzoic |
| | acid 1-(3-cyclopropylmethoxy-4-difluoromethoxy-phenyl)-2- |
| | (3,5-dichloro-pyridin-4-yl)-ethyl ester |
| C2 | (-)-3-Cyclopropylmethoxy-4-methanesulfonylamino-benzoic |
| | acid 1-(3-cyclopropylmethoxy-4-difluoromethoxy-phenyl)-2- |
| | (3,5-dichloro-1-oxy-pyridin-4-yl)-ethyl ester |
| C3 | (-)-4-Cyclopropylmethoxy-3-methanesulfonylamino-benzoic |
| | acid 1-(3-cyclopropylmethoxy-4-difluoromethoxy-phenyl)-2- |
| | (3,5-dichloro-1-oxy-pyridin-4-yl)-ethyl ester |
| C4 | (-)-3,4-Bis-methanesulfonylamino-benzoic acid 1-(3- |
| | cyclopropylmethoxy-4-difluoromethoxy-phenyl)-2-(3,5- |
| | dichloro-1-oxy-pyridin-4-yl)-ethyl ester |
| C5 | (-)-3-Methanesulfonylamino-4-methyl-benzoic acid 1-(3- |
| | cyclopropylmethoxy-4-difluoromethoxy-phenyl)-2-(3,5- |
| | dichloro-1-oxy-pyridin-4-yl)-ethyl ester |
| C6 | (-)-4-Methanesulfonylamino-3-methyl-benzoic acid 1-(3- |
| | cyclopropylmethoxy-4-difluoromethoxy-phenyl)-2-(3,5- |
| | dichloro-1-oxy-pyridin-4-yl)-ethyl ester |

The preferred compound according to the invention is (−)-3-cyclopropylmethoxy-4-methanesulfonylamino-benzoic acid 1-(3-cyclopropylmethoxy-4-difluoromethoxy-phenyl)-2-(3,5-dichloro-1-oxy-pyridin-4-yl)-ethyl ester indentified as C2.

The combinations according to the present invention comprise:

a compound of general formula (I) and a retinoid;

a compound of general formula (I) and an arsenic derivative; or all the active substances, i.e. a compound of general formula (I), a retinoid and an arsenic derivative.

Examples of retinoids to be used according to the invention can be found in Beard et al., Handbook of experimental pharmacology, retinoids the biochemical and molecular of vitamin A and retinoid action; Nau, H, Blaner, W. S. Eds.; Springer: Berlin Heidelberg 1999, vol. 139, p. 185, which is incorporated herein by reference in its entireyt).

The retinoid might be advantageously selected from the group consisting of all-trans retinoic acid, 9-cis retinoic acid or 13-cis retinoic acid, vitamin A (retinol), or carotene, more preferably from the group consisting of all-trans retinoic acid, 9-cis retinoic acid or 13-cis retinoic acid. All-trans retinoic acid, also known as ATRA, is particularly preferred.

The arsenic derivative is advantageously selected from the group consisting of arsenic disulfide, arsenic trisulfide, and arsenic trioxide. Arsenic trioxide, also known as ATO, is preferred.

Preferred combinations comprise (−)-3-cyclopropylmethoxy-4-methanesulfonylamino-benzoic acid 1-(3-cyclopropylmethoxy-4-difluoromethoxy-phenyl)-2-(3,5-dichloro-1-oxy-pyridin-4-yl)-ethyl ester (C2), all-trans retinoic acid (ATRA), and/or arsenic trioxide (ATO).

The active substances of the combination may be administered sequentially, separately or simultaneously. Advantageously, when the two or three active substances are administered together, they are administered as a fixed combination.

Therefore, the invention also provides a medicament comprising the two or three active substances as a fixed combination. The medicament may be in form of pharmaceutical composition, optionally in admixture with one or more pharmaceutically acceptable carriers or diluents, for example those described in Remington's Pharmaceutical Sciences Handbook, XVII Ed., Mack Pub., N.Y., U.S.A., which is incorporated herein by reference in its entirety.

Examples include diluents (such as sucrose, mannitol, lactose, starches) and known excipients, including suspending agents, solubilizers, buffering agents, binders, disintegrants, preservatives, colorants, flavours, lubricants and the like.

Administration of the combination of the present invention may be accomplished according to patient needs, for example, orally, parenterally, e.g. subcutaneously, intravenously, intramuscularly, and by infusion.

Various solid oral dosage forms may be used for administering the combination of the present invention including such solid forms as tablets, gelcaps, capsules, caplets, granules, lozenges, and bulk powders.

Various liquid oral dosage forms may also be used for administering the combination of the present invention, including aqueous and non-aqueous solutions, emulsions, suspensions, syrups, and elixirs. Such dosage forms can also contain known suitable inert diluents such as water and known suitable excipients such as preservatives, wetting agents, sweeteners, flavors, as well as agents for emulsifying and/or suspending the compounds of the invention. The compounds of the invention may be injected, for example, intravenously, in the form of an isotonic sterile solution. Other known preparations are also possible.

In certain preferred embodiments, the combinations of the invention might be administered together, intravenously, or orally.

In other embodiments, when the active substances are formulated separately, each individual active substance could be administered separately, intravenously or orally. In this case, the individual active substances do not unconditionally have to be taken at the same time.

In the case of such a separate administration, the formulation of the individual active substances could be packed at the same time in a suitable container means. Such separate packaging of the components in a suitable container mean is also described as a kit.

The dosages of the active substances in the combination of the present invention may depend upon a variety of factors including the particular disease to be treated, the severity of the symptoms, the route of administration, the frequency of the dosage interval, the particular compound utilized, the efficacy, toxicology profile, and pharmacokinetic profile of the compound.

Advantageously, when used, the retinoid, e.g. ATRA, may be administered at a daily dose of 0.6 to 600 mg corresponding to about 0.01 to 10 mg/kg body weight, preferably 3 to 300 mg, corresponding to about 0.05 to 5 mg/kg body weight. In some embodiments, for example for oral administration, the daily dose may be 15 to 90 mg, corresponding to about 0.25 to 1.5 mg/kg body weight, preferably 30 to 60 mg, corresponding to about 0.5 to 1.0 mg/kg body weight.

For example, ATRA is usually orally administered at a dose of 20 mg three times a day for an overall daily dose of 60 mg corresponding to about 1.0 mg/kg body weight.

ATO instead is usually administered orally at a daily dose of 0.15 mg/kg body weight.

The daily dosage of ATRA may be 0.01 to 10 mg/kg body weight, preferably 0.05 to 5 mg/kg body weight, more preferably 0.1 to 0.8 mg/kg body weight, even more preferably 0.4 to 0.6 mg/kg body weight; while that of the arsenic derivative, e.g. ATO, may be 0.01 to 1.0 mg/kg body weight, preferably 0.02 to 0.5 mg/kg body weight, more preferably 0.05 to 0.1 mg/kg body weight.

It has been found that the usual daily dose of either ATO (0.15 mg/kg) or ATRA (about 1.0 mg/kg body weight) may be reduced by up to 50 percent when combined with compounds of general formula (I).

In particular, since has it been demonstrated that a combination of half the conventional dose of ATRA with arsenic trioxide significantly reduces, in patients with AP, early mortality caused by retinoids syndrome, increases complete remission (CR) rate and reduces period to CR respect to patients treated with conventional doses of ATRA, a great advantage derives from a combination of a compound of general formula (I), a ATRA administered at a daily dose of about 0.4 to 0.6 mg/kg body weight, and ATO administered at a daily dose of about 0.05 to 0.1 mg/kg body weight.

More preferably, all the active substances are administered orally.

Advantageously, the compounds of general formula (I) may be administered at a daily dose of 0.1 to 6000 microg corresponding to about 0.0016 to 100 microg/kg body weight, preferably 120 to 3000 microg, corresponding to about 2 to 50 microg/kg body weight. In some embodiments, for example for oral administration, the daily dose may be 480 to 2400 microg, corresponding to about 8 to 40 microg/kg body weight, while in other embodiments, the daily dose may be 240 to 1200 microg, corresponding to about 4 to 20 microg/kg body weight. In further embodiments, for example for intravenous administration, the daily dose may be 0.1 to 12 microg, corresponding to about 0.0016 to 0.2 microg/kg body weight.

As reported in Example 7, the compound C2, representative of the compounds of formula (I), turned out to be capable of inducing the expressions of the early myeloid markers CD38 and CD11b in ATO-sensitive or ATO-resistant acute promyelocytic leukemia cells, thereby suggesting that this compound, when utilized as mono-treatment, is able to partially induce an early myeloid differentiation in acute promyelocytic leukemia cells.

Therefore, the present invention is also directed to the use of a compound of formula (I) for the treatment hematological malignancies.

On the basis of the findings herewith described, the skilled person could recognize that the compounds of formula (I) alone or within the combination of the invention are suitable to treat any type of lymphocytic or myeloid leukemia, either acute or chronic, in particular of leukemia refractory to the treatment with common chemotherapeutic agents.

More particularly, the combination of a compound of formula (I) with a retinoid or an arsenic derivative would turn out to be useful for the treatment of acute promyelocytic leukemia, more in particular for the treatment of the form refractory/relapsed to ATRA. Instead, the combination of a compound of formula (I) with an arsenic derivative would turn out to be particularly useful for the treatment of chronic myeloid leukemia, more in particular for the treatment of the form relapsed/refractory to Imatinib.

Other features of the invention will become apparent in the course of the following descriptions of exemplary embodiments which are given for illustration of the invention and are not intended to be limiting thereof.

### EXAMPLES

### Experimental Part

The following active substances were used: the compound C2, Imatinib mesylate (hereinafter Imatinib), roflumilast also quoted hereinafter with the code CHF 5152 and piclamilast also quoted hereinafter with the code CHF 5859.

The parental sensitive cell lines NB4, K562, LAMA and KCL22 were obtained from commercial sources (ATCC Manassas, Va., USA, and DSMZ GmbH, Braunschweig, Germany). The arsenic-resistant NB4-AsR subline was obtained by treating parental cells NB4 with ATO 1 μM weakly and then maintained with the same dose (see Lunghi P et al., Leukemia 2005; 19(2):234-44, which is incorporated herein by reference in its entirety). Imatinib resistant cell lines K562-RES, LAMA-RES and KCL22-RES were generated by exposing parental cell lines to gradually increasing doses of Imatinib and then maintained in 1 μM Imatinib (see Mahon F X et al., Blood. 2000; 96(3):1070-9, which is incorporated herein by reference in its entirety). The Imatinib resistant BaF3-p210-T315I cell line was established from murine BaF3 cells by stable transfection with plasmid expressing Bcr-Abl-T315I mutation (see La Rosée P et al., Cancer Res. 2002; 62(24):7149-53, which is incorporated herein by reference in its entirety).

Values are mean±SD of 3 independent experiments.

For multiple comparisons a statistical analysis was performed using analysis of variance for repeated measurements followed by Dunnet post-tests using JMP version 7.0 statistical software (SAS Institute, Cary, N.C.).

### Example 1

### C2 Potentiates the Proapoptotic Effects of Arsenic Trioxide in Acute Promyelocytic Leukemia Cells

The Parental Acute Promyelocytic Leukemia (APL) cell line NB4 and the Arsenic resistant NB4-AsR cell line were seeded at 0.8×10⁵ cells/mL of fresh RPMI 1640 medium (Sigma Aldrich), supplemented with 10% fetal calf serum, 2 mM L-glutamine, penicillin G (100 U/mL), streptomycin (100 mg/mL). Cells were pre-treated for 30 minutes with vehicle (DMSO), CHF-6001 (1 and 10 μM), Roflumilast (1 and 10 μM) or Piclamilast (1, and 50 μM) and then incubated with ATO at the indicated doses. After 72 hours cells were collected, stained with Annexin-V FITC (according to the manufacturer's recommendations—Bender MedSystems) and processed by flow cytometry (FACSCalibur, Becton Dickinson, San Josè, Calif.) to evaluate the percentage of apoptosis as described in Lunghi P et al., Leukemia. 2005; 19(2):234-44, which is incorporated herein by reference in its entirety.

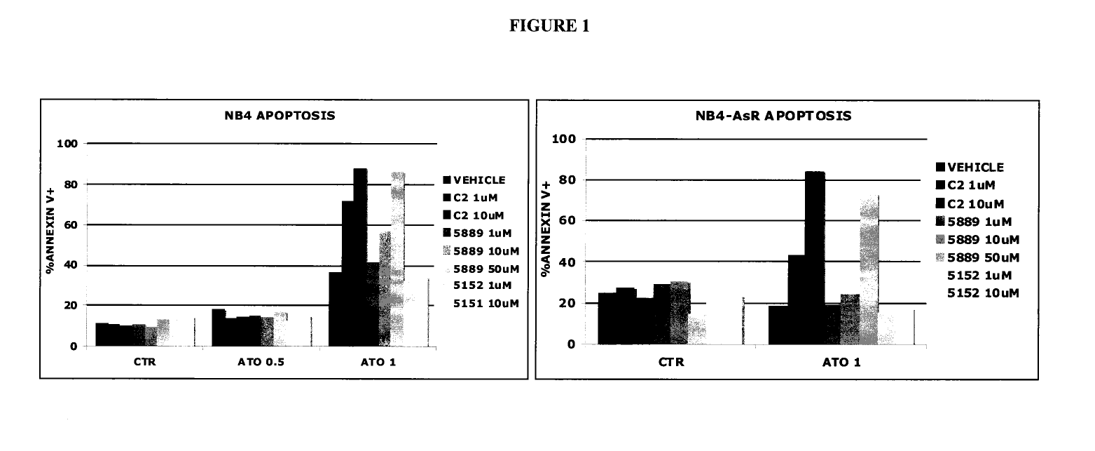

As it can be appreciated from FIG. 1, C2 potentiates the proapoptotic effects of ATO in APL cells (−P<0.001 C2 1 μM/10 μM+ATO 1 μM versus either mono-treatment). Moreover, C2 is more effective than CHF 5152 (roflumilast) or CHF 5859 (piclamilast) in potentiating the proapoptotic effects of ATO (−P<0.001 C2 1 μM/10 μM+ATO 1 μM versus same doses of CHF 5889+ATO or CHF 5152+ATO; −P<0.005 C2 1 μM+ATO 1 μM versus CHF 5889 10 μM+ATO or CHF 5152 10 μM+ATO).

### Example 2

### C2 Potentiates the Proapoptotic Effects of Arsenic Trioxide in Human Chronic Myelogenous Leukemia Cell Lines

The Imatinib-sensitive (K562-SENS, LAMA-SENS, KCL22-SENS) and Imatinib-resistant (K562-RES, LAMA-RES, KCL22-RES and BAF3 p210-T315I) chronic myeloid leukemia (CML) cell lines were cultured with the PDE-4 inhibitor and ATO as described in Example 1. After 72 hours cells were harvested, stained with Propidium Iodide to evaluate the percentage of cells with hypodiploid DNA content (sub-G1) or with Annexin-V FITC as described in Lunghi P et al Leukemia. 2005; 19(2):234-44, which is incorporated herein by reference in its entirety.

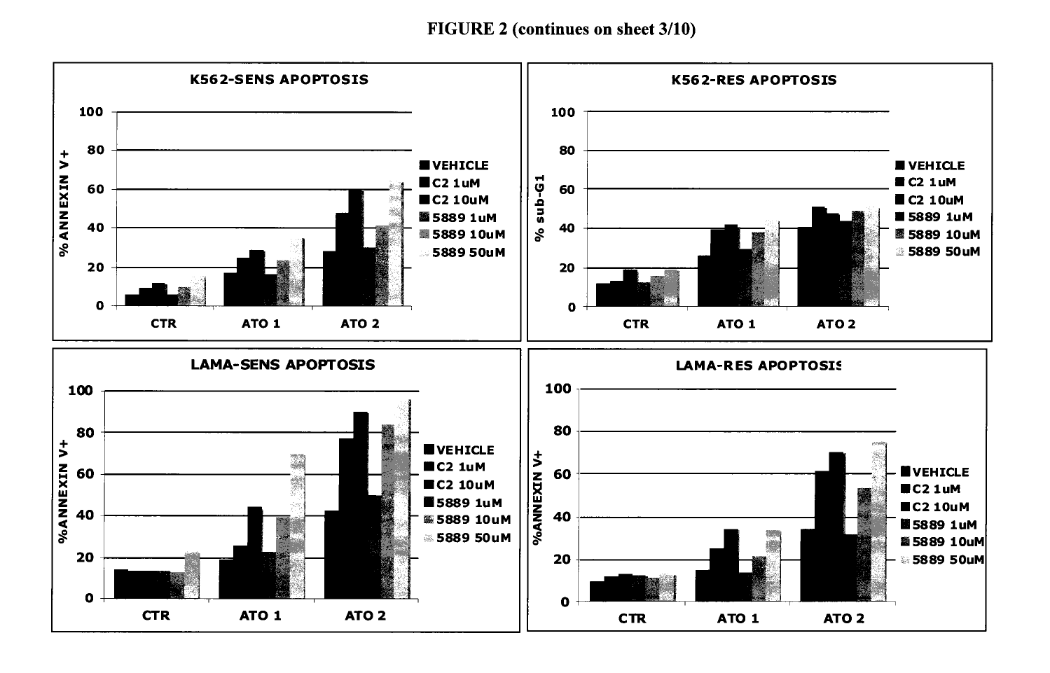

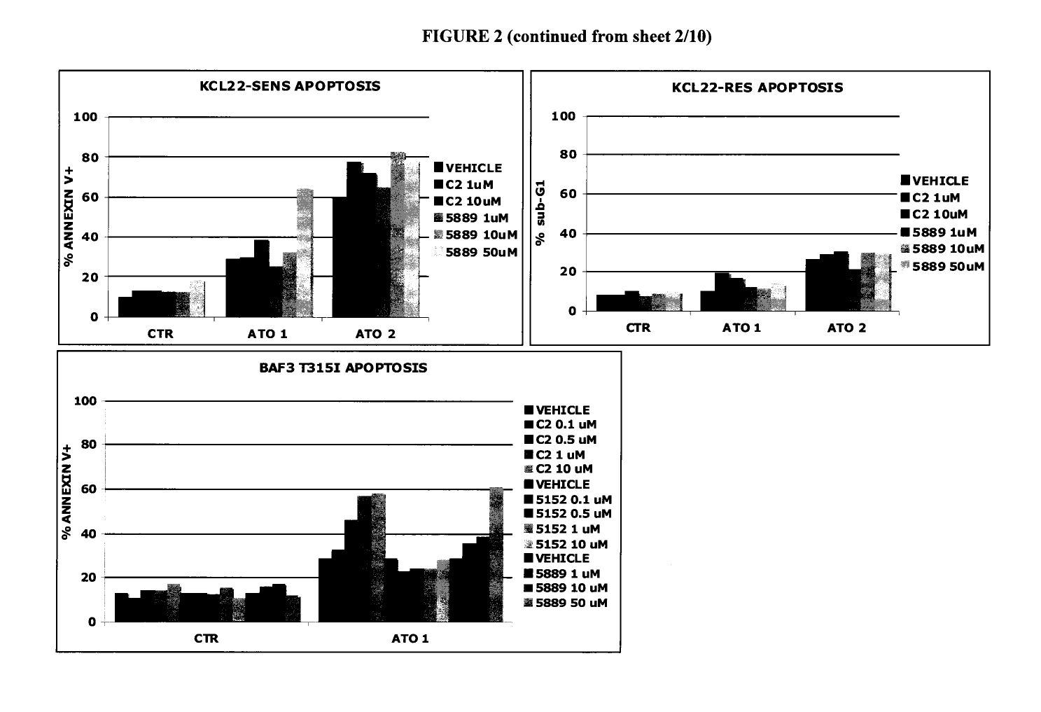

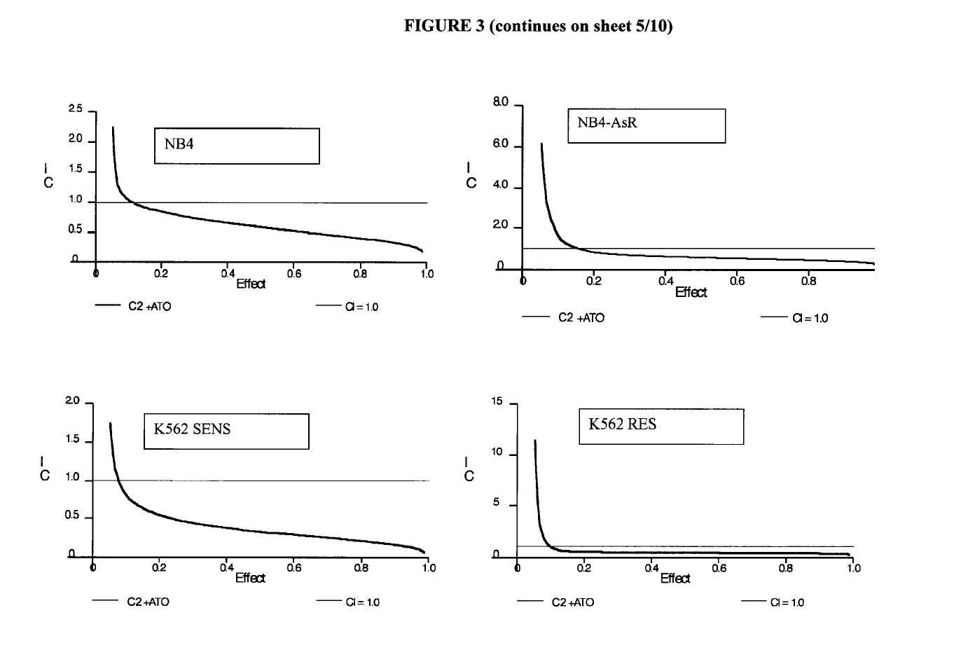

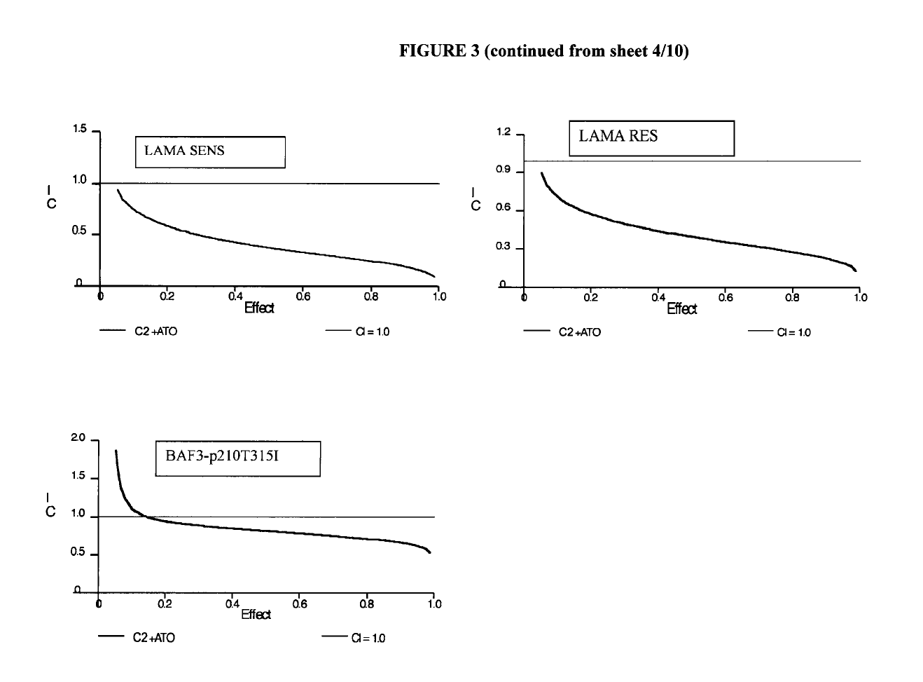

The results are reported in FIG. 2. C2 potentiates the proapoptotic effects of ATO in Human Chronic Myelogenous Leukemia cell lines (−P<0.001 C2 1 μM/10 μM+ATO 2 μM versus either mono-treatment in K562-SENS, LAMA-SENS, LAMA-RES and Baf3-T315I (ATO 1 μM); −P<0.05 C2 1 μM/10 μM+ATO 2 μM versus either mono-treatment in K562-RES). Also in this case, C2 turned out to be more effective than Roflumilast or Piclamilast in potentiating the proapoptotic effects of ATO (−P<0.001 C2 1 μM+ATO 2 μM versus same dose of CHF 5889+ATO or CHF 5152+ATO in K562-SENS, LAMA-SENS, LAMA-RES, Baf3-T315I (ATO 1 μM); −P<0.05 C2 1 μM+ATO 2 μM versus same dose of CHF 5889+ATO in K562-RES; −P<0.01 C2 10 μM+ATO 2 μM versus same dose of CHF 5889+ATO or CHF 5152+ATO in K562-SENS, LAMA-RES, Baf3-T315I (ATO 1 μM; −P<0.001 C2 1 μM+ATO 1 μM versus CHF 5889 10 μM+ATO and CHF 5152 10 μM+ATO in Baf3-T315I).

### Example 3

### C2 Synergizes with ATO to Induce Apoptosis in APL and CML Cell Lines

APL (NB4 and NB4-AsR) and CML (K562-SENS, K562-RES, LAMA-SENS, LAMA-RES and BAF3 p210-T3151) cell lines seeded at 0.8×10⁵ cells/mL were treated sequentially with escalating doses of CHF-6001 (0.5-10 μM) for 30 minutes and subsequently with ATO (0.5-5 μM) alone or in combination with CHF-6001 at a fixed ratio 1:1 (0.5/0.5, 1/1, 1.5/1.5, 2/2 μM). After 72 hours, cells were harvested and apoptosis was measured as percentage of cells with hypodiploid DNA content by Propidium Iodide staining and flow cytometry. Combination index (CI) plots was then generated using the Chou-Talalay method and Calcusyn software (Biosoft, Ferguson, Mo.). CI values lesser than 1.0 indicates synergism; CI value equal to 1.0 indicates additive effect; CI more than 1.0 indicates antagonistic effect (Lunghi P et al., Leukemia. 2005; 19(2):234-44, which is incorporated herein by reference in its entirety). The results, reported in FIG. 2, shows that the combine treatment of C2 plus ATO gives rise to a synergistic induction of apoptosis in APL and CML cell lines.

### Example 4

### Caspase Activity is Indispensable in C2/ATO-Induced Apoptosis

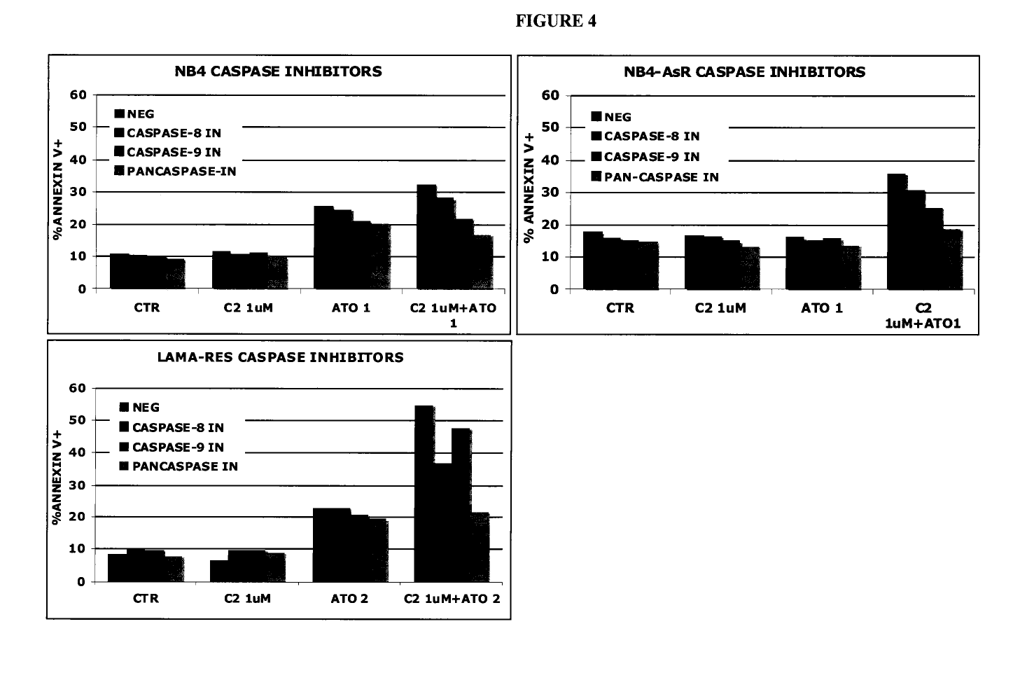

LAMA-RES, NB4 and NB4-AsR cell lines were pretreated with selective caspase inhibitors for 1 hour before adding C2 and/or ATO. After 24 hours the percentage of apoptotic cells was determined by the annexin V method. More specifically, the caspase inhibitors are: NEG peptide control Cbz-Phe-Ala-fluoromethyl ketone (Z-FA-FMK), CASPASE-8 inhibitor Cbz-Ile-Glu(Ome)-Thr-Asp(Ome)-fluoromethyl ketone (Z-IETD-FMK), CASPASE-9 inhibitor Cbz-Leu-Glu(Ome)-His-Asp(Ome)-fluoromethyl ketone (Z-LEHD-FMK), and the PANCASPASE inhibitor Cbz-Val-Ala-Asp(Ome)-fluoromethyl ketone (Z-VAD-FMK), all provided from Alexis (San Diego, Calif., USA). All caspase inhibitors were dissolved in DMSO, stocked in aliquots at −20° C., and used at the final concentration of 25 μM. FIG. 4 shows that C2 strikingly enhances ATO-induced cytotoxicity in leukemia cells through a caspase-dependent mechanism.

### Example 5

### The Combination C2/ATO Affects the Apoptotic Pathway

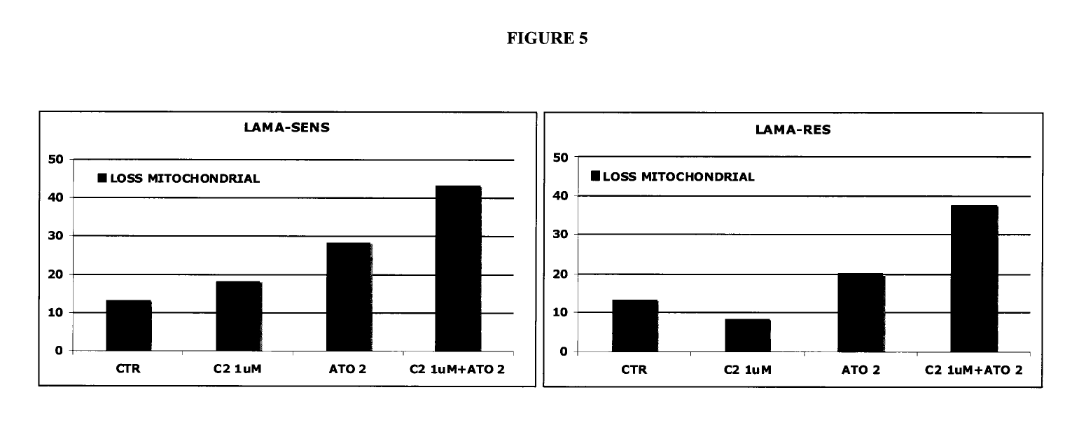

LAMA-SENS and LAMA-RES cell lines were cultured with C2/ATO for 72 hours, after which cells were harvested for determination of the loss of mitochondrial transmembrane potential ΔΨm by flow cytometry using MitoCapture Apoptosis Detection kit (BioVision). This kit is a fluorescent-based method for distinguishing between healthy and apoptotic cells by detecting the changes in the mitochondrial transmembrane potential. The kit utilizes a cationic dye that fluoresces differently in healthy (red fluorescence) versus apoptotic cells (green fluorescence). The results in FIG. 5 demonstrate that the combination C2/ATO activates the mitochondrial apoptotic pathway.

### Example 6

### C2 Potentiates the Cytodifferentiating Action of Retinoids and Arsenic Trioxide in Acute Promyelocytic Leukemia Cells

NB4 and NB4-AsR were seeded at 0.8×10⁵ cells/mL in presence of vehicle (DMSO) or the PDE-4 inhibitor at the indicated doses for 30 minutes and then incubated with 0.1 μM and 1 μM of all-trans retinoic acid (ATRA) or with low-doses ATO (0.5 μM) promoting cell differentiation (see Chen GQ et al., Blood. 1997; 89(9):3345-53, which is incorporated herein by reference in its entirety). After 72 hours of treatment, early and late stage of differentiation was evaluated. More specifically, expression of the cell surface CD11b, an early-expressed maturation marker, was measured by flow cytometry using fluorescein isothiocyanate (FITC)-conjugated anti-CD11b antibody (Sigma Chemical Co.) according to the manufacturer's protocol. Late stage differentiation of leukemic cells was detected using the microscopic nitroblue tetrazolium (NBT) reduction test (Chen GQ et al., vide supra).

This assay is conducted by counting the cells containing blue NBT formazan deposits, which are formed by reduction of the membrane permeable, water-soluble, yellow-colored, nitroblue tetrazolium (NBT) by superoxide anions O2. Over 300 cells were counted per sample, and variation in replicates was routinely within 10%.

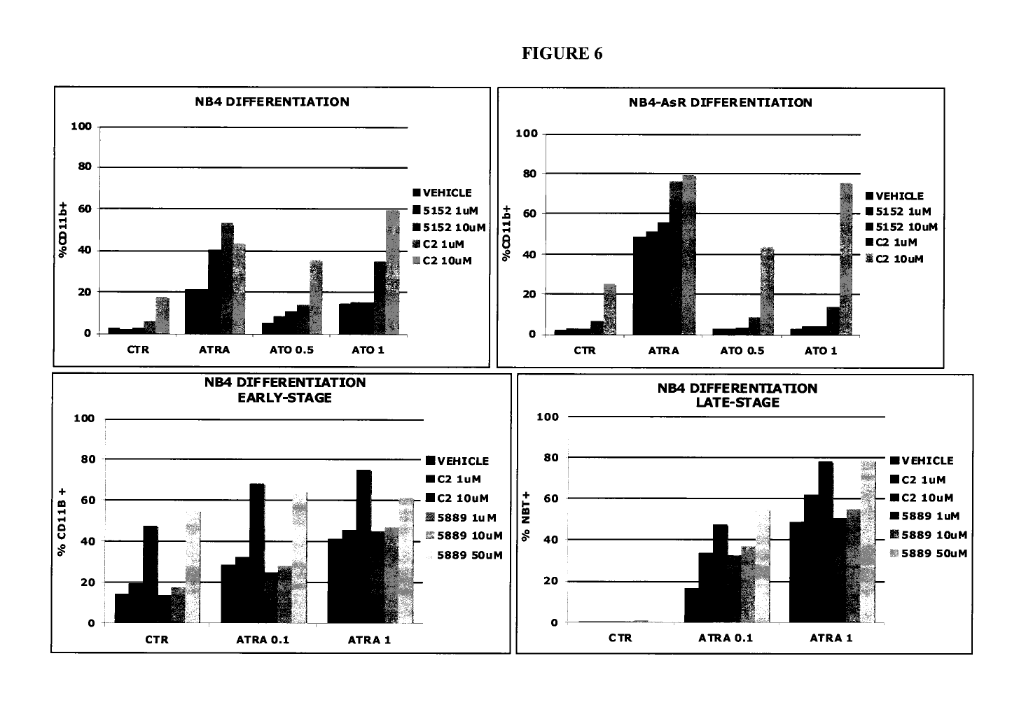

The results shown in FIG. 6 indicate that C2 upregulates the expression of the myeloid marker CD11b, indicating that it potentiates the cytodifferentiating action of ATRA and ATO in APL Cells (−P<0.001 C2 1 μM/10 μM+ATRA and C2 1 μM/10 μM+ATO 1 μM versus either mono-treatment in NB4; −P<0.001 C2 1 μM/10 μM+ATRA and C2 10 μM+ATO 1 μM versus either mono-treatment in NB4-AsR).

Also in this case, C2 is more effective than Roflumilast or Piclamilast in potentiating the cytodifferentiating action of ATRA or ATO in ATO-sensitive or ATO-resistant APL Cell lines (−P<0.001 C2 1 μM+ATRA versus same dose of CHF 5152+ATO in NB4 and NB4-AsR, −P<0.001 C2 10 μM+ATRA versus same dose of CHF 5152+ATO in NB4-AsR; −P<0.001 C2 10 μM+ATO 1 μM versus same dose of CHF 5152+ATO in NB4 and NB4-AsR, −P<0.001 C2 1 μM+ATO 1 μM versus same dose of CHF 5152+ATO in NB4; −P<0.001 C2 10 μM+ATRA 0.1 μM/1 μM versus same dose of CHF 5889+ATRA in Early-Stage differentiation of NB4; −P<0.001 C2 10 μM+ATRA 1 μM versus same dose of CHF 5889+ATRA in Late-Stage differentiation of NB4, −P<0.01 C2 10 μM+ATRA 0.1 μM versus same dose of CHF 5889+ATRA in Late-Stage differentiation of NB4).

### Example 7

### C2 Enhances Cytodifferentiating Properties of Retinoids in Acute Promyelocytic Leukemia Cells

NB4 e NB4-AsR cell lines were cultured with C2/ATRA for 72 hours, after which cells were harvested for determination of NBT reduction and expression of differentiation-associated surface antigens CD33, CD38, CD11b, CD14 and CD15. Cytofluorimetric analysis of surface antigen expression was performed using the following monoclonal antibodies all provided by Sigma Chemical: fluorescein isothiocyanate (FITC)-conjugated anti-CD33 antibody, (FITC)-conjugated anti-CD38 antibody, (FITC)-conjugated anti-CD11b antibody, (FITC)-conjugated anti-CD 14 antibody and (FITC)-conjugated anti-CD15 antibody. Briefly, each antibody was incubated at the proper dilution with cell samples in PBS containing 1% BSA for 30 minutes at room temperature. Cells were then washed and resuspended with PBS and analyzed by flow cytometer.

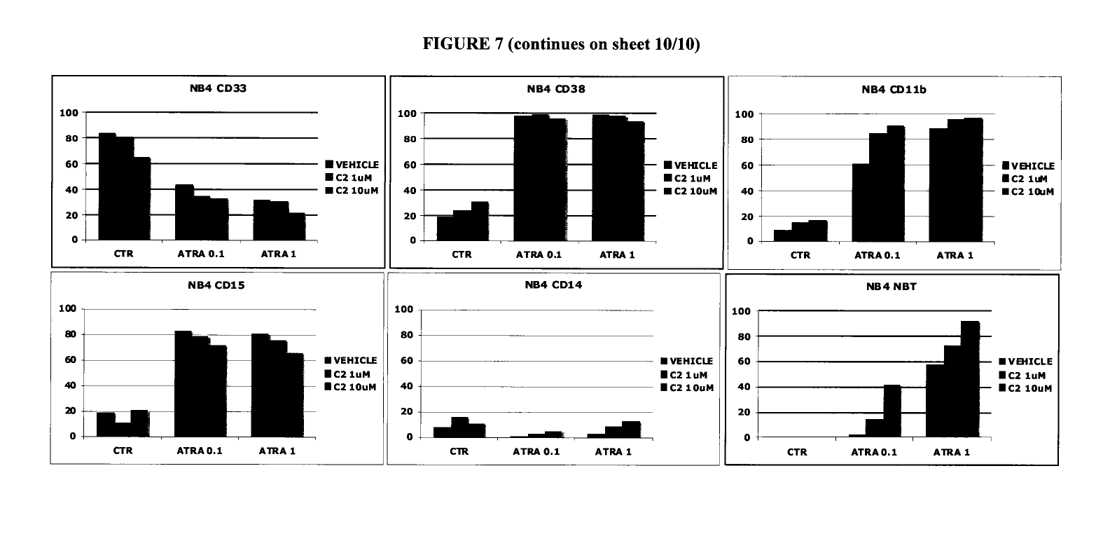

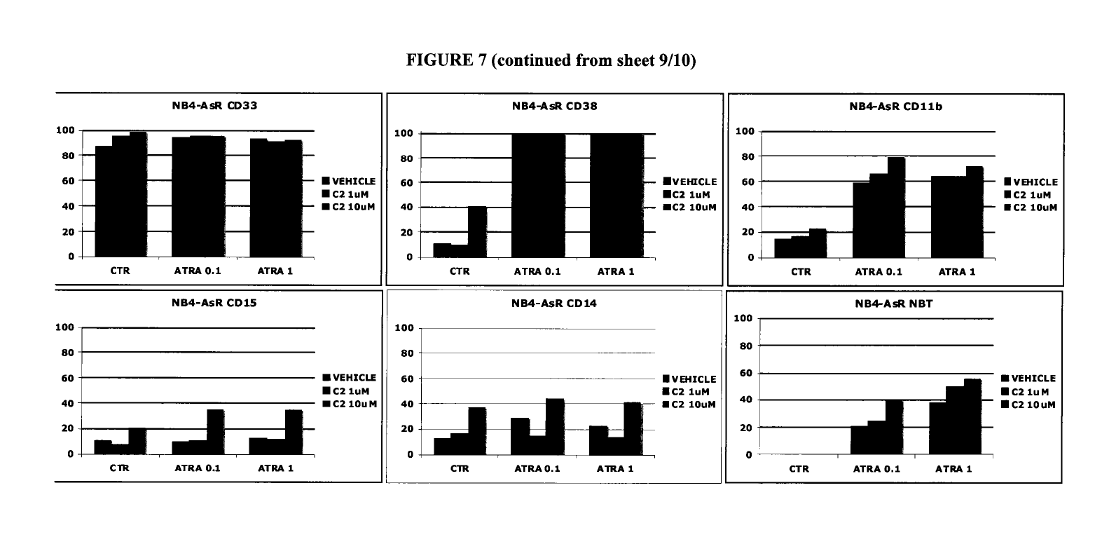

From FIG. 7, it can be appreciated that C2 induces upregulation of the myeloid markers (CD38 and CD11b) and enhances the cytodifferentiating action of very low-dose of ATRA in ATO-sensitive or ATO-resistant APL Cell lines.

Furthermore C2, at doses 5-fold lower than piclamilast, is able to induce the expressions of the early myeloid markers CD38 and CD 11b in ATO-sensitive or ATO-resistant acute promyelocytic leukemia cells, thereby suggesting that this compound, when utilized as mono-treatment, is able to partially induce an early myeloid differentiation in acute promyelocytic leukemia cells.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A combination comprising:
(A) at least one compound of formula (I) or a pharmaceutically acceptable salt thereof: 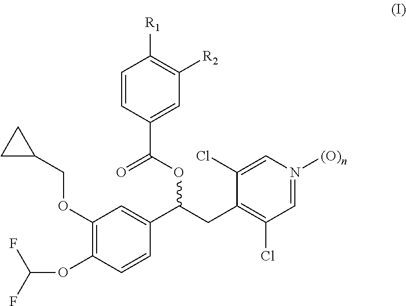
wherein:
n is 0 or 1;
R₁ and R₂, which may be the same or different, are independently:
linear or branched (C₁-C₆)alkyl, optionally substituted by one or more halogen atoms;
OR₃, wherein R₃ is a linear or branched (C₁-C₆)alkyl group optionally substituted with one or more halogen atoms or C₃-C₇ cycloalkyl groups; or
HNSO₂R₄, wherein R₄ is a linear or branched (C₁-C₄)alkyl group optionally substituted with one or more halogen atoms,
wherein at least one of R₁ and R₂ is HNSO₂R₄; and
(B) at least one of:
(i) at least one retinoid and/or
(ii) at least one arsenic derivative.

2. A combination according to claim 1, wherein said compound of formula (I) is selected from the group consisting of:
(−)-3-cyclopropylmethoxy-4-methanesulfonylamino-benzoic acid 1-(3-cyclopropylmethoxy-4-difluoromethoxy-phenyl)-2-(3,5-dichloro-pyridin-4-yl)-ethyl ester;
(−)-3-cyclopropylmethoxy-4-methanesulfonylamino-benzoic acid 1-(3-cyclopropylmethoxy-4-difluoromethoxy-phenyl)-2-(3,5-dichloro-1-oxy-pyridin-4-yl)-ethyl ester;
(−)-4-cyclopropylmethoxy-3-methanesulfonylamino-benzoic acid 1-(3-cyclopropylmethoxy-4-difluoromethoxy-phenyl)-2-(3,5-dichloro-1-oxy-pyridin-4-yl)-ethyl ester;
(−)-3,4-bis-methanesulfonylamino-benzoic acid 1-(3-cyclopropyl-methoxy-4-difluoromethoxy-phenyl)-2-(3,5-dichloro-1-oxy-pyridin-4-yl)-ethyl ester;
(−)-3-methanesulfonylamino-4-methyl-benzoic acid 1-(3-cyclopropyl-methoxy-4-difluoromethoxy-phenyl)-2-(3,5-dichloro-1-oxy-pyridin-4-yl)-ethyl ester; and
(−)-4-methanesulfonylamino-3-methyl-benzoic acid 1-(3-cyclopropylmethoxy-4-difluoromethoxy-phenyl)-2-(3,5-dichloro-1-oxy-pyridin-4-yl)-ethyl ester;

3. A combination according to claim 2, wherein said compound of formula (I) is (−)-3-cyclopropylmethoxy-4-methanesulfonylamino-benzoic acid 1-(3-cyclopropylmethoxy-4-difluoromethoxy-phenyl)-2-(3,5-dichloro-1-oxy-pyridin-4-yl)-ethyl ester.

4. A combination according to claim 1, wherein said arsenic derivative is selected from the group consisting of arsenic disulfide, arsenic trisulfide, and arsenic trioxide.

5. A combination according to claim 4, wherein said arsenic derivative is arsenic trioxide (ATO).

6. A combination according to claim 5, wherein said arsenic trioxide is present in an amount to result in administration of a daily dose of 0.01 to 1.0 mg/kg body weight.

7. A combination according to claim 1, wherein said retinoid is selected from the group consisting of all-trans retinoic acid, 9-cis retinoic acid, 13-cis retinoic acid, vitamin A (retinol), and carotene.

8. A combination according to claim 7, wherein said retinoid is all-trans retinoic acid (ATRA).

9. A combination according to claim 8, wherein all-trans retinoic acid is present in an amount to result in administration of a daily dose of 0.01 to 10 mg/kg body weight.

10. A combination according to claim 1, wherein said retinoid is all-trans retinoic acid and is present in an amount to result in administration of a daily dose of about 0.4 to 0.6 mg/kg body weight, and said arsenic derivative is arsenic trioxide and is present in an amount to result in administration of a daily dose of about 0.05 to 0.1 mg/kg body weight.

11. A medicament, comprising a fixed combination of at least one compound of formula (I) as defined in claim 1, and either at least one retinoid and/or at least one arsenic derivative, and optionally a pharmaceutically acceptable carrier or diluent.

12. A kit comprising;
a) a compound of formula (I) as defined in claim 1, and optionally a pharmaceutically acceptable carrier or diluent in a first unit dosage form;
b) a retinoid and optionally a pharmaceutically acceptable carrier or diluent in a second unit dosage form; and/or
c) an arsenic derivative, and optionally a pharmaceutically acceptable carrier or diluent in a third unit dosage form;
d) container means for containing said first, second and optionally third dosage forms.

13. A method for the treatment of a hematological malignancy, comprising administering an effective amount of at least one compound of formula (I) or a pharmaceutically acceptable salt thereof: 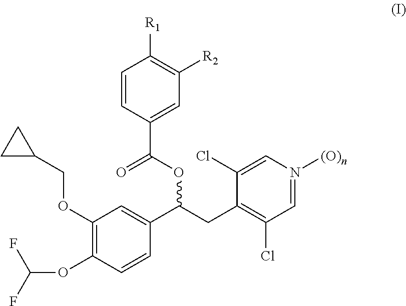
wherein:
n is 0 or 1;
R₁ and R₂, which may be the same or different, are independently:
linear or branched (C₁-C₆)alkyl, optionally substituted by one or more halogen atoms;
OR₃, wherein R₃ is a linear or branched (C₁-C₆)alkyl group optionally substituted with one or more halogen atoms or C₃-C₇ cycloalkyl groups; or
HNSO₂R₄, wherein R₄ is a linear or branched (C₁-C₄)alkyl group optionally substituted with one or more halogen atoms,
wherein at least one of R₁ and R₂ is HNSO₂R₄;
to a subject in need thereof.

14. A method according to claim 13, wherein said compound of formula (I) is selected from the group consisting of:
(−)-3-cyclopropylmethoxy-4-methanesulfonylamino-benzoic acid 1-(3-cyclopropylmethoxy-4-difluoromethoxy-phenyl)-2-(3,5-dichloro-pyridin-4-yl)-ethyl ester;
(−)-3-cyclopropylmethoxy-4-methanesulfonylamino-benzoic acid 1-(3-cyclopropylmethoxy-4-difluoromethoxy-phenyl)-2-(3,5-dichloro-1-oxy-pyridin-4-yl)-ethyl ester;
(−)-4-cyclopropylmethoxy-3-methanesulfonylamino-benzoic acid 1-(3-cyclopropylmethoxy-4-difluoromethoxy-phenyl)-2-(3,5-dichloro-1-oxy-pyridin-4-yl)-ethyl ester;
(−)-3,4-bis-methanesulfonylamino-benzoic acid 1-(3-cyclopropyl-methoxy-4-difluoromethoxy-phenyl)-2-(3,5-dichloro-1-oxy-pyridin-4-yl)-ethyl ester;
(−)-3-methanesulfonylamino-4-methyl-benzoic acid 1-(3-cyclopropyl-methoxy-4-difluoromethoxy-phenyl)-2-(3,5-dichloro-1-oxy-pyridin-4-yl)-ethyl ester; and
(−)-4-methanesulfonylamino-3-methyl-benzoic acid 1-(3-cyclopropylmethoxy-4-difluoromethoxy-phenyl)-2-(3,5-dichloro-1-oxy-pyridin-4-yl)-ethyl ester;

15. A method according to claim 13, wherein said compound of formula (I) is (−)-3-cyclopropylmethoxy-4-methanesulfonylamino-benzoic acid 1-(3-cyclopropylmethoxy-4-difluoromethoxy-phenyl)-2-(3,5-dichloro-1-oxy-pyridin-4-yl)-ethyl ester.

16. A method according to claim 13, wherein said malignancy is lymphocytic or myeloid leukemia, either acute or chronic.

17. A method according to claim 13, wherein the myeloid leukemia form is acute promyelocytic leukemia.

18. A method according to claim 13, further comprising administering at least one retinoid to said subject in need thereof.

19. A method according to claim 13, further comprising administering at least one arsenic compound to said subject in need thereof.

20. A method according to claim 13, further comprising administering at least one retinoid and at least one arsenic compound to said subject in need thereof.
